(19) United States
(12) Patent Application Publication
(10) Pub. No.: US 2009/0266910 A1
(43) Pub. Date: Oct. 29, 2009

(54) Fragrance Filter Air Escape Patch For Plastic Trash Bags

(76) Inventor: James Hunter, Cumming, GA (US)

Correspondence Address: DONALD W. MEEKER, 924 EAST OCEAN FRONT, # E, NEWPORT BEACH, CA, 92661, US

(21) Appl. No.: 12/111,139

(22) Filed: Apr. 28, 2008

(51) Int. Cl. A61L 9/04; B65D 33/01

(52) U.S. Cl. 239/57; 383/103

(57) ABSTRACT

A trash liner system comprises a scented, ventilated patch for air to escape from between the bag and receptacle. The patch adheres over a hole near the top of the bag. Escaping air passing through the patch is scented with the fragrance contained in the patch.

### CROSS-REFERENCE TO RELATED APPLICATIONS

Not Applicable.

### STATEMENT REGARDING FEDERALLY SPONSORED RESEARCH OR DEVELOPMENT

Not Applicable.

### THE NAMES OF THE PARTIES TO A JOINT RESEARCH OR DEVELOPMENT

Not Applicable.

### BACKGROUND OF THE INVENTION

1. Field of the Invention

The present invention relates to garbage bags and particularly to a flow-through fragrance filter patch which is mounted in a venting aperture of trash bag, which allows for release of air trapped between the trash bag and the trash receptacle and which scents the escaping air.

2. Description of Related Art Including Information Disclosed Under 37 CFR 1.97 and 1.98

Most trash receptacles are lined with plastic garbage bags; however a problem occurs when filling the trash bag in that the air trapped between the bag and the receptacle prevents the bag from being filled completely, as shown in FIG. 1. When the plastic trash bag is inserted into a trash can and the bag is stretched over its top rim, air is trapped between the can and bag. When garbage is loaded into the plastic bag, the trapped air can prevent filling the bag to more than 50 percent capacity. A user must release the trapped air by removing the bag from the top rim of the can and re-stretching the bag over the top rim. These actions are time consuming and the released air is often unpleasant. Vented trash cans and vented retaining clamps or rings for the bags have been proposed as a solution to the problem, but often the bag retainers take as much time to employ as releasing the bag from the top of the can and vented trash cans present a mess if the plastic bag leaks.

Conventional trash bags do not provide an effective means for controlling odors. Some plastic bags are impregnated with odor eating compositions or have fragrances added to the plastic during the manufacturing process, but this does not help with the problem of trapped air between the plastic bag and the trash receptacle.

U.S. Patent Application #20070248291, published Oct. 25, 2007 by Reeves, indicates a vented plastic bag which is adapted for containing a powdery product. The plastic bag includes a plurality of walls joined together and formed of a flexible plastic material. The walls define a fill opening for receiving the powdery product into the plastic bag. At least one of the bag walls defines a ventilation zone including a plurality of spaced-apart perforations. A filter medium overlies the ventilation zone and covers the plurality of perforations to substantially capture and retain the powdery product within the plastic bag during fill and ventilation.

U.S. Pat. No. 6,135,637, issued Oct. 24, 2000 to Kim, claims a compressible disposal plastic trash sack having series of vertical air slots made up of fine perforations on upper portions of both sack walls that break open from stress of compression to allow excess air inside sack to discharge to outside atmosphere. The discharge of air facilitates sack to compact upon its self and flexible trash contents within, further minimizing sack volume and continually maximizing receptacle holding capacity for the duration which compression is generated by weight of other sacks or applied by persons. U.S. Pat. No. 5,213,141, issued May 25, 1993 to Dorman, describes a debris collection and disposal system for collection and transfer of lawn debris consists of a vented collapsible bag and a rigid container, for structure for releasably inter-engaging them in mouth-to-mouth relationship.

U.S. Pat. No. 2,092,969, issued Sep. 14, 1937 to Gustafson, indicates a vented garbage can liner in which apertures are provided in the liner to vent the air between the liner and the receptacle.

U.S. Pat. No. 6,808,073, issued Oct. 26, 2004 to Cuisinier, shows a bag assembly with a connection means for temporarily binding nesting bags to each other and preferably for later additionally acting as a closure means for those same bags. The assembly is configured such that an adhesive is revealed when a nested bag is removed from the assembly. That same adhesive then provides a closure means for the orifice of the bag. Furthermore, the bag body, and identical bag bodies, may possess air holes. Air holes are positioned and structured to allow trapped air between the nested bag bodies and trapped air between the bag assembly and the support device to escape.

U.S. Pat. No. 4,978,231, issued Dec. 18, 1990 to Ling, puts forth a multiple disposable plastic bag assembly comprised of multiple disposable plastic bags nested one within another to form a bag assembly, characterized in that sticking elements are made on the circular top edge of the opening of the bag body for sticking of the bag assembly to a wastebin to allow the disposable plastic bag assembly to be torn up consecutively bag by bag so as to minimize time consumption in placing individual bags in a wastebin and to facilitate packing up garbage within a bag of the assembly. The two opposite large surfaces of the bag body are provided with a plurality of fine air holes to prevent air collection between the wastebin and the bag body so as to facilitate placing of the bag assembly in a wastebin. The fine air holes are in the form of arcuate slits.

U.S. Pat. No. D525,877, issued Aug. 1, 2006 to Wingfield, concerns the ornamental design for an air-vented tieless trash bag with elastic cord.

U.S. Pat. No. 6,003,717, issued Dec. 21, 1999 to Long, illustrates a combination of loop-tie trash liner in combination with a trash container, and a method of using them to dispose of waste material. In the preferred embodiment, there is a notch in the edge of the opening of the bag, between two openings of the tubular channel, whereby air may be vented when the bag is placed inside the trash container. The trash liner is a bag having an opening and a body without perforations, with the opening of the bag having an edge within which is a tubular channel, and a flexible tying member passing through the tubular channel. Opposite ends of the tying member extend from openings in the tubular channel. The trash liner may be fastened on a trash container by inserting the body of the bag into the trash container, placing the edge of the opening of the bag over the rim of the trash container, and tying the opposite ends of the tying member together. Trash placed within the trash container may be safely removed by untying the ends of the tying member, removing the edge of the opening of the bag from the rim of the trash container, closing the opening of the bag, and again tying the ends of the tying member to secure closure of the bag.

U.S. Patent Application #20070020410, published Jan. 25, 2007 by Sasine, describes a method and process of collecting, packaging and processing recyclable waste which shows, in FIG. 5, plastic bags that include one or more air release holes. The release holes are formed near the neck of bags and are configured to allow air to be easily released from bags when bags are compacted by a baler.

U.S. Patent Application #20050101207, published May 12, 2005 by Bonnechere, claims a fragrance pad which comprises a water insoluble substrate that is impregnated with a fragrance solution, wherein an adhesive means is provided for affixing the water insoluble substrate to the interior surface of a garbage bag.

U.S. Pat. No. 5,119,968, issued Jun. 9, 1992 to Palmer, is for a scented trash bag dispenser is comprised of a plurality of disposable trash bags arranged in an ordered stack and connected by a frangible connection to a base member. The base member is connectable to a separate object or surface to suspend the stacked plurality of trash bags from the object or surface, where the forward most trash bag of the plurality of bags is easily accessible for use. Either or both of the base member and plurality of trash bags are constructed of a material that emits an air freshening scent to perfume the air in the vicinity of the trash bag dispenser.

U.S. Pat. No. 4,349,104, issued Sep. 14, 1982 to Hayes, provides a scented disposal bag which is used for disposal of odorous material such as, but not limited to, sanitary napkins, tampons and the like. A scented or deodorizing material is selectively released within the bag when the bag is closed.

U.S. Patent Application #20050201643, published Sep. 15, 2005 by Ulloa-Parra, discloses a scented disposable trash bag includes a body having an open top end portion and a closed bottom end portion. The body defines a cavity extending between the top and bottom end portions and includes a flexible outer surface impregnated with a pesticide. The outer surface further includes a predetermined quantity of fragrance substance impregnated therein for emitting a pleasant aroma. The body further includes a plurality of flexible straps having opposed end portions attached to the body. The plurality of straps are connected adjacent the top end portion of the body and are engageable with each other for tying the open top end portion.

What is needed is an air release opening through the bag adjacent to the top to allow air to escape from between a trash container and the bag to allow the bag to fill completely to the limits of the size of the container, with a fragrance filter patch over the air release opening for imparting a pleasant fragrance to the air.

### BRIEF SUMMARY OF THE INVENTION

An object of the present invention is to provide an air release opening through the bag adjacent to the top to allow air to escape from between a trash container and the bag to allow the bag to fill completely to the limits of the size of the container, with a fragrance filter patch over the air release opening for imparting a pleasant fragrance to the air.

Another object of the present invention is that different fragrances may be made available.

One more object of the present invention is that bags may be produced with patches in place over pre-cut holes, or patches may be produced to be used on standard bags a consumer would already have.

In brief, the present invention provides a trash liner system that ventilates the air between bag and receptacle through a scented patch near the top of the bag to provide a means for air to escape, allowing the bag to be filled to capacity and a providing a pleasant fragrance to mask the smell of trash.

The purpose of the device is to provide a trash liner bag with a scented, ventilated patch to allow the bag to be filled to capacity and a supply a pleasant aroma.

An advantage of the present invention is that the bag is easy to put in place.

Another advantage of the present invention is that the ventilated patch allows the air to pass and not get trapped between the can and bag so the bag can be filled to capacity.

One more advantage of the present invention is the patches on the bag provide a pleasant fragrance.

An additional advantage of the present invention is that many different fragrances may be available.

A further advantage of the present invention is that patches may be sold separately and easily put on existing bags by merely cutting a hole in the existing bag, transforming any bag into a Fragrance Filter Patch Bag.

A contributory advantage of the present invention is bags may be mass produced and quantities packaged together for sale.

### BRIEF DESCRIPTION OF THE SEVERAL VIEWS OF THE DRAWINGS

These and other details of my invention will be described in connection with the accompanying drawings, which are furnished only by way of illustration and not in limitation of the invention, and in which drawings:

FIG. 1 is a cross-sectional view taken through a prior art trash container with an air impermeable prior art trash bag which traps air between the trash container and the bag with the top of the bag sealed around the top rim of the trash container;

FIG. 2 is a cross-sectional view of the fragrant air release filter patch of the present invention taken through the air release filter patch;

FIG. 3 is a cross-sectional view of the fragrant air release filter patch of the present invention taken through the trash container and the trash bag not through the air release filter patch so that the patch is visible in elevation;

FIG. 4 is an elevational view of the fragrant air release filter patch showing the central filter material and the adhesive outer border;

FIG. 5 is a partial elevational view of a trash bag showing an air release filter patch of the present invention adhered to the outside of the bag over a hole (shown dashed) through the bag.

### DETAILED DESCRIPTION OF THE INVENTION

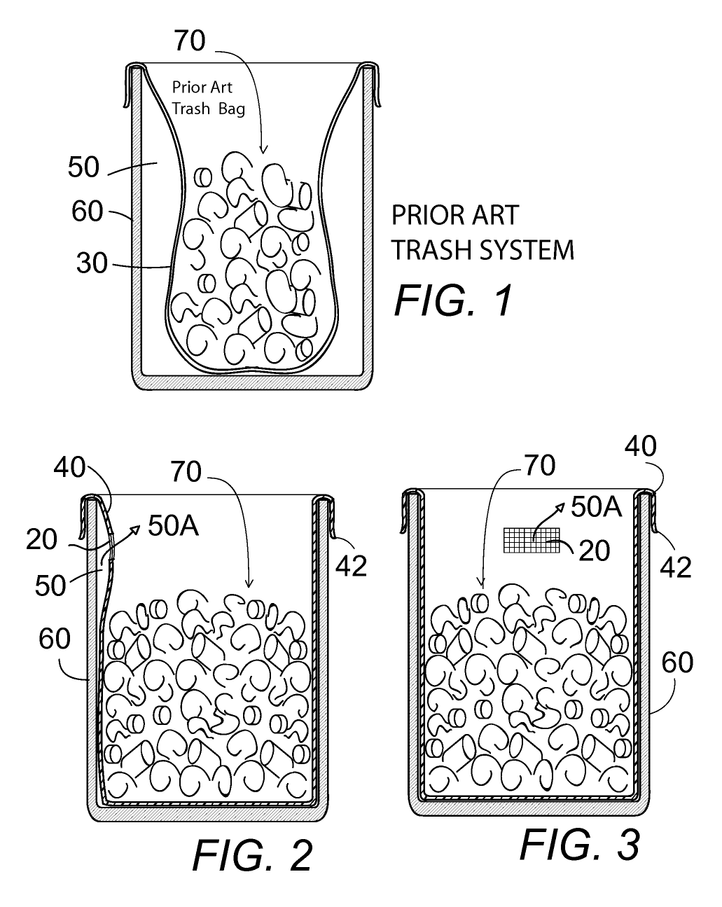

In FIG. 1, a prior art trash container with an air impermeable prior art trash bag 40 traps air 50 between the trash container 60 and the bag 40 with the top of the bag sealed around the top rim of the trash container.

In FIGS. 2-5, a vented fragrant air release filter device 10 for a trash bag 40 comprises a patch 20 formed of an air release filter material 21 impregnated with a scent to produce a fragrance in air 50A passing through the filter material and an adhesive border 22 for adhering the patch 20 to the bag 40 over an air release hole 41 through the bag near the top.

In FIGS. 2-5, a patch 20 adheres to a plastic trash bag 40 over a hole 41 through a side of the trash bag 40 spaced near a top opening in the trash bag. The patch comprises a flat strip of air permeable filter material 21 impregnated with a scented substance for imparting a fragrance to the air 50A passing through the filter material 21 and an outer border 22 around the filter material having an adhesive on one face of the border to adhere the patch 20 to the bag 40 around the hole 41 for filtering all of the air passing through the hole. The patch 20 is positioned so that with the bag 40 inserted in a trash container and the edge 42 of the top opening in the bag draped over a top rim around a trash container 60 opening, the patch releases air 50A from between the trash container and the bag 40, so that the bag can be filled completely to the total size of the trash container interior with no trapped air between the bag and the trash container and impart a fragrance to the air passing through the filter material.

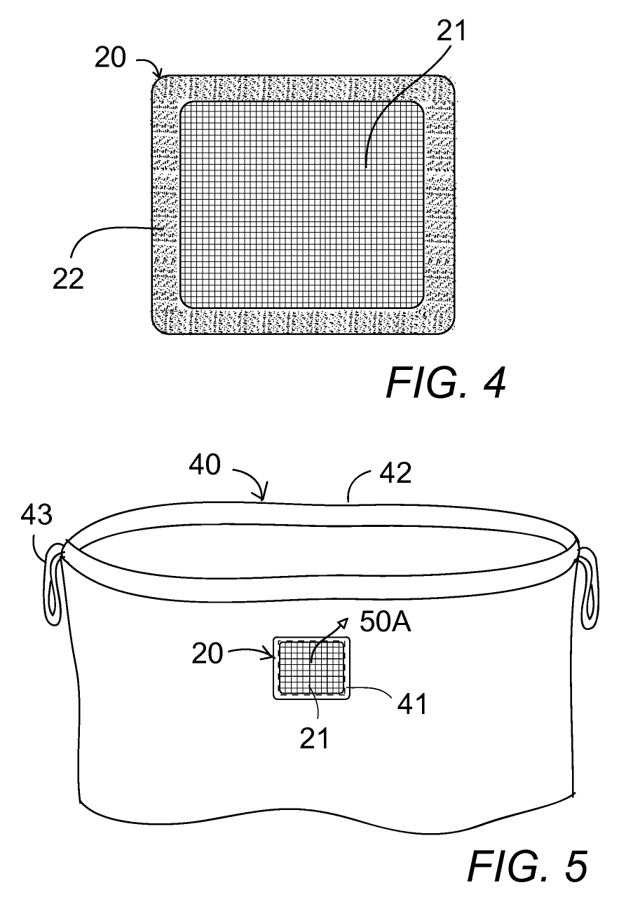

In FIGS. 4 & 5, the patch 20 comprises a sheet of adhesive material adhered to the filter material 21 with the sheet of adhesive being larger in area than the filter material so that the outer perimeter of the adhesive sheet overlaps the filter material to form the adhesive outer border 22. The adhesive sheet contains openings therethrough permitting the filter material to admit the released air therethrough.

The patch further comprises a peel-off cover strip over the adhesive border so that the cover strip is removable to adhere the patch over a hole cut in an existing trash bag. The hole through the bag 41 is cut and the patch adhered over the hole in a production run of a number of bags and a quantity of the bags containing patches are packaged together.

In use, with the Fragrance Filter Patch Bags of the present invention air escapes from between the bag and the can through the Fragrance Filter Patches so the trash can gets completely filled easily without air between the bag and the can to prevent the trash from filling the can. The air is scented by passing through the Scented Filter Patch to cover up the trash smell for a more pleasant experience in filling or emptying the trash can. New Hunter Fragrance Filter Patch Bags have a single Fragrance Filter Patch near the top of the bag to allow the air to escape from between the can and the bag and to scent the air at the same time with any of a variety of scents imparted to the air by the filters which are impregnated with a scent.

It is understood that the preceding description is given merely by way of illustration and not in limitation of the invention and that various modifications may be made thereto without departing from the spirit of the invention as claimed.

What is claimed is:

1. A vented fragrant air release filter device for a trash bag, the device comprising:
a patch for adhering to a plastic trash bag over a hole through a side of the trash bag spaced near a top opening in the trash bag, the patch comprising a flat strip of air permeable filter material impregnated with a scented substance for imparting a fragrance to air passing through the filter material and an outer border around the filter material bearing an adhesive on one face of the outer border to adhere the patch to the bag around the hole for filtering all of the air passing through the hole, the patch positioned so that with the bag inserted in a trash container and an edge of the top opening in the bag draped over a top rim around a trash container opening, the patch releases air from between the trash container and the bag so that the bag can be filled completely to the total size of the trash container interior with no trapped air between the bag and the trash container, the patch imparting a fragrance to the air passing through the filter material.

2. The device of claim 1 wherein the patch comprises a sheet of adhesive material adhered to the filter material, the sheet of adhesive larger in area than the filter material so that the outer perimeter of the adhesive sheet overlaps the filter material to form the adhesive outer border, the adhesive sheet having openings therethrough over the filter material to admit the released air therethrough.

3. The device of claim 1 wherein the patch further comprises a peel-off cover strip over the adhesive border so that the cover strip is removable to adhere the patch over a hole cut in an existing trash bag.

4. The device of claim 1 wherein the hole through the bag is cut and the patch adhered over the hole in a production run of a number of bags and a quantity of the bags with patches packaged together.
